(19) United States
(12) Patent Application Publication
(10) Pub. No.: US 2015/0080338 A1
(43) Pub. Date: Mar. 19, 2015

(54) COSMETIC COMPOSITION COMPRISING A SILANE AND A LIPOPHILIC THICKENER

(71) Applicant: L'OREAL, Paris (FR)

(72) Inventors: Raluca Lorant, Thiais (FR); Laetitia Feuillette, Paris (FR)

(21) Appl. No.: 14/395,890

(22) PCT Filed: Apr. 24, 2013
(86) PCT No.: PCT/EP2013/058518

§ 371 Date: Oct. 21, 2014

Related U.S. Application Data
(60) Provisional application No. 61/674,027, filed on Jul. 20, 2012.

(30) Foreign Application Priority Data
Apr. 26, 2012 (FR) 1253870

(51) Int. Cl. A61K 8/58; A61Q 5/00; A61K 8/81; A61K 8/86; A61K 8/34; A61K 8/891; A61Q 19/00; A61K 8/37

(52) U.S. Cl. CPC A61K 8/585; A61Q 19/007; A61Q 19/001; A61Q 5/00; A61K 8/375; A61K 8/86; A61K 8/37; A61K 8/342; A61K 8/891; A61K 8/8158; A61K 2800/48
USPC 514/63

(57) ABSTRACT

The present invention relates to a composition comprising at least one lipophilic thickener and at least one silane corresponding to formula (I) below and/or oligomers thereof: R₁Si(OR₂)z(R₃)ₓ(OH)_{y} (I) in which: *R₁ is a linear or branched, saturated or unsaturated C₁-C₂₂ hydrocarbon-based chain, which may be substituted with a hydroxyl group, a thiol group, an aryl group (more particularly benzyl), which is substituted or unsubstituted; R₁ possibly being interrupted with a heteroatom (O or S) or a carbonyl group (CO), *R₂ and R₃, which may be identical or different, represent a linear or branched alkyl group comprising from 1 to 6 carbon atoms, *y denotes an integer ranging from 0 to 3, and *z denotes an integer ranging from 0 to 3, and *x denotes an integer ranging from 0 to 2, * with z+x+y=3, the silane(s) of formula (I) and/or oligomers thereof being present in an active material content ranging from 2% to 20% by weight relative to the total weight of the composition. The invention also relates to a cosmetic process for making up and/or caring for keratin materials comprising a step of applying a composition according to one of the preceding claims to the said materials.

The invention relates to a cosmetic composition intended in particular for keratin materials, especially for the skin, the lips, the hair and the nails. The invention also relates to a cosmetic process for treating keratin materials using the said composition.

A recurrent problem in the field of cosmetic haircare consists in caring for keratin fibres subjected to various external attacking factors. Specifically, these fibres may be subject to attacking factors of various origins, such as mechanical attack, for example linked to disentangling or blow-drying, or alternatively chemical attack, for example following dyeing or permanent-waving.

These attacking factors have consequences on the qualities of the keratin fibre and may lead to difficult disentangling at the time of washing the hair, on dry hair and/or wet hair, and also to degradation of the surface properties of the fibres, which become non-smooth and irregular at the surface, more particularly when the hair is dry.

Cosmetic haircare compositions exist that can limit these phenomena. In particular, it is known practice to use silanes in order to improve the condition of the hair.

International patent application WO 2004/012 691, for example, discloses the cosmetic use of silanes for improving the condition of the hair.

Document EP 0 159 628 proposes compositions for reinforcing the elasticity of the hair, comprising an alkyltrialkoxysilane.

Moreover, document EP 1 736 139 describes a hair treatment composition comprising an alkoxysilane, an organic acid and water, the pH of the composition being between 2 and 5.

Finally, patent application EP 0 877 027 discloses a composition comprising an organosilane and a particular polyol.

Among the known silanes, fatty-chain silanes are efficient for affording haircare. However, they are difficult to use, which is reflected by instability of the cosmetic compositions and/or poor availability of these silanes by interaction with the other ingredients of the cosmetic composition, resulting in a reduction of their efficiency.

Thus, there is a need to develop novel formulatory systems that enable satisfactory use of fatty-chain silanes while otherwise maintaining good working properties.

In the cosmetic field, and more particularly in the field of skincare and photoprotection, it is known practice to use galenical architectures containing a fatty phase containing lipophilic thickeners. This makes it possible to improve the efficacy and the stability of the products. Moreover, the use of these compounds makes it possible to improve the quality of the deposit on the skin.

In particular, in the field of photoprotection, the use of solid fatty substances in the fatty phase containing lipophilic screening agents makes it possible to improve the homogeneity of the protective film and the SPF while at the same time improving the resistance to water and to sand.

Moreover, structuring of the fatty phase with fatty substances is particularly advantageous for giving consistency to anhydrous or emulsified compositions intended for caring for or making up the skin or for haircare. Solid fatty substances are very often used to obtain a thickening effect and more or less “solid” galenical forms—between balms and butters and ranging up to galenical forms in stick form (such as lipsticks).

Nevertheless, this type of preparation has the drawback of being difficult to apply and/or of depositing heterogeneous films and leaving an unpleasant greasy and shiny residual film—these products are especially accused of producing a “tacky” effect on application and after penetration. Furthermore, the finish on the skin is often sparingly glidant—i.e. the skin does not feel soft, but, on the contrary, has a “dragging” effect. In the case of haircare, these compositions often produce a greasy feel which transfers onto the hands, also giving an impression of hair that is greasy, or even dirty.

There is thus still a need to produce compositions containing a fatty phase, which would not have these drawbacks but would enable pleasant and easy spreading on the skin or the hair, while at the same time leaving a silky finish, without leaving a greasy film at the surface of the skin or the hair.

The Applicant has found, surprisingly, that the combination of a particular silane and of a lipophilic thickener makes it possible to improve the sensory properties of cosmetic compositions containing them, by allowing a glidant and “fondant” effect on application, which induces easy, pleasant and homogeneous application and a softer skin quality after penetration into the skin.

Similarly, the spreading on keratin fibres is easy and treated hair has a smooth, soft and non-greasy feel. In particular, the combination according to the invention allows hold of the hairstyle without producing a greasy look or feel.

More specifically, one subject of the present invention is a cosmetic composition comprising:

at least one lipophilic thickener and

at least one silane of formula (I) below and/or oligomers thereof:

R₁Si(OR₂)_{z}(R₃)ₓ(OH)_{y} (I)

in which:
R₁ is a linear or branched, saturated or unsaturated C₁-C₂₂, C₃-C₄₀ cycloalkyl or C₆-C₃₀ aromatic hydrocarbon-based chain; optionally substituted with a hydroxyl group, a thiol group, an aryl group (more particularly benzyl), which is substituted or unsubstituted; R₁ possibly being interrupted with a heteroatom (O, S or NH) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
with z+x+y=3,
   the silane(s) of formula (I) and/or oligomers thereof being present in an active material content ranging from 2% to 20% by weight relative to the total weight of the composition.

Another subject of the invention consists of a composition that may be obtained from one or more lipophilic thickeners and from one or more silanes of formula (I) and/or oligomers thereof.

A subject of the invention is also the use of the said composition in cosmetics or dermatology, and in particular for caring for, protecting and/or making up bodily or facial skin, or for haircare.

A subject of the invention is also a cosmetic process for treating keratin materials, in which a cosmetic composition as defined previously is applied to these keratin materials.

In that which follows, the expression “at least one” is equivalent to “one or more” and, unless otherwise indicated, the limits of a range of values are included within this range.

The composition according to the invention is intended for application to keratin materials and thus comprises a physiologically acceptable medium. The term “physiologically acceptable medium” is understood here to mean a medium that is compatible with keratin materials, such as the skin, mucous membranes, the scalp, the eyes and/or keratin fibres such as the eyelashes or the hair.

### Lipophilic Thickener:

The composition according to the invention comprises at least one lipophilic thickener.

According to the present invention, the term “thickener” means compounds which, by their presence, increase the viscosity of the fatty phase into which they are introduced by at least 20 cps and preferably by at least 50 cps, at 25° C. and at a shear rate of 1 s⁻¹ (the viscosity may be measured using a cone/plate viscometer, a Haake R600 rheometer or the like).

This thickener may be chosen from semi-crystalline polymers, mineral lipophilic thickeners, organic lipophilic thickeners other than semi-crystalline polymers, and mixtures thereof.

For the purposes of the invention, the term “semi-crystalline polymer” means a polymer comprising a crystallizable portion, pendent chain or block in the backbone, and an amorphous portion in the backbone and having a first-order reversible temperature of phase change, in particular of melting (solid-liquid transition). When the crystallizable portion is a block of the polymer backbone, this crystallizable block has a chemical nature different from that of the amorphous blocks; in this case, the semi-crystalline polymer is a block polymer, for example of the diblock, triblock or multiblock type.

Advantageously, the semi-crystalline polymer(s) of the composition of the invention have a number-average molecular mass M̅n of greater than or equal to 2000, for example ranging from 2000 to 800 000, preferably from 3000 to 500 000, for example from 4000 to 150 000 and better still from 4000 to 99 000.

In the composition according to the invention, the semi-crystalline polymers are advantageously soluble in the oily phase, to at least 1% by weight, at a temperature that is higher than their melting point. Apart from the crystallizable chains or blocks, the blocks of the polymers are amorphous. For the purposes of the invention, the expression “crystallizable chain or block” means a chain or block which, if it were alone, would change from the amorphous state to the crystalline state reversibly, depending on whether one is above or below the melting point. For the purposes of the invention, a “chain” is a group of atoms, which is pendent or lateral relative to the polymer backbone. A “block” is a group of atoms belonging to the backbone, this group constituting one of the repeating units of the polymer.

Preferably, the polymer backbone of the semi-crystalline polymers is soluble in the oily phase.

Preferably, the semi-crystalline polymers used in the composition of the invention have a melting point, m.p., of less than 70° C. (25° C.≦m.p.<70° C.), this temperature being at least equal to the temperature of the keratin material that is to receive the composition according to the invention, especially the skin. The melting point may especially be measured by any known method and in particular with a differential scanning calorimeter (DSC).

Preferably, the crystallizable blocks or chains of the semi-crystalline polymers represent at least 30% of the total weight of each polymer and better still at least 40%. The semi-crystalline polymers bearing crystallizable blocks used according to the invention are block or multiblock polymers. They may be obtained via polymerization of monomers containing reactive double bonds (or ethylenic bonds) or via polycondensation. When the polymers of the invention are polymers bearing crystallizable side chains, they are advantageously in random or statistical form.

The semi-crystalline polymers of the invention are of synthetic origin. Moreover, they do not comprise a polysaccharide backbone.

The semi-crystalline polymers that may be used in the invention are preferably chosen from polymers (homopolymers or copolymers) bearing at least one crystallizable side chain and polymers (homopolymers or copolymers) bearing in the backbone at least one crystallizable block, such as those described in document U.S. Pat. No. 5,156,911. The crystallizable side chain(s) or block(s) are hydrophobic.

According to one particular embodiment of the invention, the semi-crystalline polymers are especially chosen from homopolymers and copolymers resulting from the polymerization of at least one monomer bearing crystallizable chain(s), this chain being chosen from alkyl chains comprising at least 11 carbon atoms and not more than 40 carbon atoms and better still not more than 24 carbon atoms. They are especially alkyl chains comprising at least 12 carbon atoms, and they are preferably alkyl chains comprising from 14 to 24 carbon atoms (C₁₄-C₂₄). They may be hydrocarbon-based alkyl chains (carbon and hydrogen atoms) or fluoroalkyl or perfluoroalkyl chains (carbon atoms, fluorine atoms and possibly hydrogen atoms). When they are fluoroalkyl or perfluoroalkyl chains, they comprise at least 11 carbon atoms, at least 6 of which carbon atoms are fluorinated.

The term “alkyl” means a saturated group (not comprising any unsaturation).

According to one particular embodiment of the invention, the semi-crystalline polymer is chosen from homopolymers obtained by polymerization of at least one monomer containing a crystallizable chain, chosen from C₁₄-C₂₄ alkyl(meth)acrylates, C₁₁-C₁₅ perfluoroalkyl (meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoroalkyl chains, C₁₄ to C₂₄ α-olefins, para-alkylstyrenes with a C₁₄ to C₂₄ alkyl group, and from the copolymers of these monomers, obtained by copolymerization of these monomers with a hydrophilic monomer, preferably other than methacrylic acid, for instance N-vinylpyrrolidone, hydroxyethyl acrylate, hydroxyethyl methacrylate, acrylic acid. Such copolymers may be, for example, copolymers of C₁₄-C₂₄-alkyl acrylate, C₁₄-C₂₄-alkyl methacrylate, C₁₄-C₂₄-alkylacrylamide or C₁₄-C₂₄-alkylmethacrylamide with N-vinylpyrrolidone, hydroxyethyl acrylate, hydroxyethyl methacrylate or acrylic acid, or mixtures thereof.

Preferably, the semi-crystalline polymer is chosen from homopolymers obtained by polymerization of a monomer chosen from C₁₄-C₂₄ alkyl acrylates and C₁₄-C₂₄ alkyl methacrylates and from copolymers obtained by copolymerization of a monomer chosen from C₁₄-C₂₄ alkyl acrylates and C₁₄-C₂₄ alkyl methacrylates with a hydrophilic monomer such as acrylic acid.

The semi-crystalline polymers in the composition of the invention may or may not be partially crosslinked, provided that the degree of crosslinking does not interfere with their dissolution or dispersion in the oily phase by heating above their melting point. It may then be a case of chemical crosslinking, by reaction with a multifunctional monomer during the polymerization. It may also be a case of physical crosslinking, which may then be due either to the establishment of bonds of hydrogen or dipolar type between groups borne by the polymer, for instance dipolar interactions between carboxylate ionomers, these interactions being in small amount and borne by the polymer backbone; or due to a phase separation between the crystallizable blocks and the amorphous blocks borne by the polymer.

Preferably, the semi-crystalline polymers of the composition according to the invention are noncrosslinked.

According to one particular embodiment of the invention, the semi-crystalline polymer is a homopolymer resulting from the polymerization of a monomer bearing a crystallizable chain chosen from C₁₄-C₂₄ alkyl acrylates and C₁₄-C₂₄ alkyl methacrylates. Mention may be made especially of those sold under the name Intelimer® by the company Landec, described in the brochure Intelimer® Polymers, Landec IP22. These polymers are in solid form at room temperature. They bear crystallizable side chains and correspond to saturated C₁₄-C₂₄ alkyl acrylate or methacrylate homopolymers. Mention may be made more particularly of the stearyl acrylate homopolymer (Intelimer IPA-13.1) (INCI name: Poly C10-30 alkyl acrylate), the behenyl acrylate homopolymer (Intelimer IPA-13.6) (INCI name: Poly C10-30 alkyl acrylate).

According to another particular embodiment of the invention, the semi-crystalline polymer is a copolymer of C₁₄-C₂₄ alkyl acrylates or of C₁₄-C₂₄ alkyl methacrylates with acrylic acid. Copolymers of this type that may be mentioned include the copolymers obtained by copolymerization of behenyl acrylate and of acrylic acid, and the copolymers obtained by copolymerization of stearyl acrylate and of acrylic acid.

According to a preferred embodiment of the invention, the semi-crystalline polymer is a homopolymer, and it is chosen from the stearyl acrylate homopolymer (Intelimer IPA-13.1) (INCI name: Poly C10-30 alkyl acrylate), the behenyl acrylate homopolymer (Intelimer IPA-13.6) (INCI name: Poly C10-30 alkyl acrylate), and mixtures thereof.

Mineral lipophilic thickeners that may be mentioned include optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is possible in particular to replace silanol groups with hydrophobic groups; a hydrophobic silica is then obtained. The hydrophobic groups can be:

trimethylsiloxyl groups, which are obtained especially by treating fumed silica in the presence of hexamethyldisilazane. Silicas thus treated are known as Silica silylate according to the CTFA (6th edition, 1995). They are sold, for example, under the references Aerosil R812® by the company Degussa, and Cab-O-Sil TS-530® by the company Cabot;

dimethylsilyloxyl or polydimethylsiloxane groups, which are obtained especially by treating fumed silica in the presence of polydimethylsiloxane or dimethyldichlorosilane. Silicas thus treated are known as “Silica dimethyl silylate” according to the CTFA (6th edition, 1995). They are sold, for example, under the references Aerosil R972® and Aerosil R974® by the company Degussa, and CAB-O-SIL TS-610® and CAB-O-SIL TS-720® by the company Cabot.

The hydrophobic fumed silica in particular has a particle size that may be nanometric to micrometric, for example ranging from about 5 to 200 nm.

The organic lipophilic thickeners other than the semi-crystalline polymers may be polymeric or non-polymeric. The polymeric lipophilic thickeners are, for example, partially or totally crosslinked elastomeric organopolysiloxanes of three-dimensional structure, for instance those sold under the names KSG6®, KSG16® and KSG18® by the company Shin-Etsu, Trefil E-505C® and Trefil E-506C® by Dow Corning, Gransil SR-CYC®, SR DMF10®, SR-DC556®, SR 5CYC gel®, SR DMF 10 gel® and SR DC 556 gel® by the company Grant Industries and SF 1204® and JK 113® by the company General Electric; ethylcellulose, for instance the product sold under the name Ethocel® by the company Dow Chemical; polycondensates of polyamide type resulting from condensation between (α) at least one acid chosen from dicarboxylic acids comprising at least 32 carbon atoms such as fatty acid dimers and (β) an alkylenediamine and in particular ethylenediamine, in which the polyamide polymer comprises at least one carboxylic acid end group esterified or amidated with at least one monoalcohol or monoamine comprising from 12 to 30 linear and saturated carbon atoms, and in particular ethylenediamine/stearyl dilinoleate copolymers such as the product sold under the name Uniclear 100 VG® by the company Arizona Chemical; silicone polyamides of the polyorganosiloxane type such as those described in documents U.S. Pat. No. 5,874,069, U.S. Pat. No. 5,919,441, U.S. Pat. No. 6,051,216 and U.S. Pat. No. 5,981,680, for instance those sold under the references Dow Corning 2-8179 and Dow Corning 2-8178 Gellant Gellant by the company Dow Corning; galactomannans comprising from one to six and in particular from two to four hydroxyl groups per saccharide, substituted with a saturated or unsaturated alkyl chain, for instance guar gum alkylated with C₁ to C₆ and in particular C₁ to C₃ alkyl chains, and mixtures thereof; block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

The compositions according to the invention may also comprise a non-emulsifying silicone elastomer as lipophilic thickener. Non-emulsifying elastomers are described especially in applications JP-A-61-194009, EP-A-242219, EP-A-285886 and EP-A-765656. Spherical non-emulsifying elastomers that may be used include those sold under the names DC9040, DC9041, DC9509, DC9505 and DC9506 by the company Dow Corning. The spherical non-emulsifying silicone elastomer may also be in the form of a powder of elastomeric crosslinked organopolysiloxane coated with silicone resin, especially with silsesquioxane resin, as described, for example, in U.S. Pat. No. 5,538,793. Such elastomers are sold under the names KSP-100, KSP-101, KSP-102, KSP-103, KSP-104 and KSP-105 by the company Shin-Etsu.

Other elastomeric crosslinked organopolysiloxanes in the form of spherical powders may be powders of a hybrid silicone functionalized with fluoroalkyl groups, sold especially under the name KSP-200 by the company Shin-Etsu; powders of a hybrid silicone functionalized with phenyl groups, sold especially under the name KSP-300 by the company Shin-Etsu.

Silicone elastomers bearing a group MQ, such as those sold by the company Wacker under the names Belsil RG100, Belsil RPG33 and, preferentially, RG80, may also be used in the compositions according to the invention. These particular elastomers, when they are in combination with the resins according to the invention, may make it possible to improve the transfer-resistance properties of the compositions comprising them.

Among the lipophilic gelling agents that may also be mentioned are organogelling agents and especially:
the bis-urea derivatives of general formula (II):

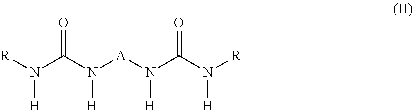

in which:
A is a group of formula:

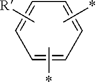

with R′ being a linear or branched C₁ to C₄ alkyl radical and the *s symbolizing the points of attachment of the group A to each of the two nitrogen atoms of the rest of the compound of general formula (II), and
R is a saturated or unsaturated, non-cyclic, mono-branched C₆ to C₁₅ alkyl radical whose hydrocarbon-based chain is optionally interrupted with 1 to 3 heteroatoms chosen from O, S and N, or
a salt or isomer thereof, described especially in patent application FR-A-2 892 303,
the silicone bis-urea derivatives of general formula (I), or a salt and/or isomer thereof:

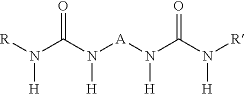

in which:
A is a group of formula (IIbis):

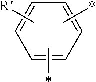

with R₁ being a linear or branched C₁ to C₄ alkyl radical and the *s symbolizing the points of attachment of the group A to each of the two nitrogen atoms of the rest of the compound of general formula (II), and
R and R′, which may be identical or different, are chosen from:
i) the radicals of formula (III):

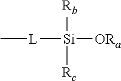

in which:
L is a single bond or a divalent carbon-based radical, especially a linear, branched and/or cyclic, saturated or unsaturated hydrocarbon-based radical (alkylene), comprising 1 to 18 carbon atoms, and possibly comprising 1 to 4 heteroatoms chosen from N, O and S;
Rₐ is:
a) a carbon-based radical, especially a linear, branched and/or cyclic, saturated or unsaturated hydrocarbon-based radical (alkyl), comprising 1 to 18 carbon atoms, and possibly comprising 1 to 8 heteroatoms chosen from N, O, Si and S; or
b) a silicone radical of formula:

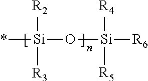

with n being between 0 and 100, especially between 1 and 80, or even 2 to 20;
and R₂ to R₆ being, independently of each other, carbon-based radicals, especially linear or branched hydrocarbon-based radicals (alkyl) containing 1 to 12 and especially 1 to 6 carbon atoms, and possibly comprising 1 to 4 heteroatoms, especially O;
R_{b} and R_{c} are, independently of each other, chosen from:
a) carbon-based radicals, especially linear, branched and/or cyclic, saturated or unsaturated hydrocarbon-based radicals (alkyl), comprising 1 to 18 carbon atoms, and possibly comprising 1 to 4 heteroatoms chosen from N, O, Si and S;
b) the radicals of formula:

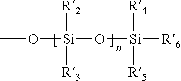

with n being between 0 and 100, especially between 1 and 80, or even 2 to 20;
and R′₂ to R′₆ being, independently of each other, carbon-based radicals, especially linear or branched hydrocarbon-based radicals (alkyl), containing 1 to 12 and especially 1 to 6 carbon atoms, and possibly comprising 1 to 4 heteroatoms, especially O;
and
ii) linear, branched and/or cyclic, saturated or unsaturated C₁-C₃₀ alkyl radicals, optionally comprising 1 to 3 heteroatoms chosen from O, S, F and N;
it being understood that at least one of the radicals R and/or R′ is of formula (III), such as those described in patent application FR-A-2 900 819,
the bis-urea derivatives described in patent application FR-A-2 894 476.

Among the lipophilic thickeners that may be used in the compositions according to the invention, mention may also be made of saccharide or polysaccharide mono- or polyalkyl esters, for instance alkyl or polyalkyl esters of dextrin or of inulin, in particular C₈-C₁₈ fatty acid esters of dextrin, such as dextrin palmitates, especially those sold under the name Rheopearl TL® or Rheopearl KL® by the company Chiba Flour.

Preferably, the organic lipophilic thickener is non-silicone, i.e. it does not contain any Si—O sequences.

According to the invention, the lipophilic thickener(s) may be present in the composition of the invention in a content ranging from 0.1% to 50% by weight, preferably from 0.5% to 30% by weight, preferably ranging from 1% to 20% by weight and preferentially ranging from 2% to 15% by weight relative to the total weight of the composition.

### Silane:

The silane(s) that may be used in the composition according to the invention are those corresponding to formula (I) below and/or oligomers thereof:

R₁Si(OR₂)_{z}(R₃)ₓ(OH)_{y} (I)

in which:
R₁ is a linear or branched, saturated or unsaturated C₁-C₂₂ hydrocarbon-based chain, which may be substituted with a hydroxyl group, a thiol group, an aryl group (more particularly benzyl), which is substituted or unsubstituted; R₁ possibly being interrupted with a heteroatom (O or S) or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
with z+x+y=3.

The term “oligomer” means the polymerization products of the compounds of formula (I) comprising from 2 to 10 silicon atoms.

Preferably, R₂ represents an alkyl group comprising from 1 to 4 carbon atoms, better still a linear alkyl group comprising from 1 to 4 carbon atoms, and preferably the ethyl group.

Preferably, R₃ represents an alkyl group comprising from 1 to 4 carbon atoms, better still a linear alkyl group comprising from 1 to 4 carbon atoms, and preferably methyl or ethyl groups.

Preferably, R₁ represents an alkyl group, and even more preferentially a linear alkyl group, comprising from 7 to 18 carbon atoms and more particularly from 7 to 12 carbon atoms or a C₁-C₆ and preferably C₂-C₄ aminoalkyl group. More particularly, R₁ represents an octyl group.

Preferably, z ranges from 1 to 3. Even more preferentially, z is equal to 3.

Preferably, the composition comprises at least one silane chosen from octyltriethoxysilane, dodecyltriethoxysilane, octadecyltriethoxysilane and hexadecyltriethoxysilane.

More particularly, the composition comprises at least octyltriethoxysilane (OTES).

The silane(s) of formula (I) and/or oligomers thereof may be present in the composition of the invention in proportions ranging from 2% to 20% by weight, preferentially ranging from 2% to 15% and better still from 5% to 15% by weight relative to the total weight of the composition.

The composition according to the invention may be aqueous or anhydrous.

The composition according to the invention may be in any galenical form conventionally used for a topical application and especially in the form of dispersions of aqueous gel or lotion type, emulsions of liquid or semi-liquid consistency of the milk type, obtained by dispersing a fatty phase in an aqueous phase (O/W) or vice versa (W/O), or suspensions or emulsions of soft, semi-solid or solid consistency of the cream or gel type, or alternatively multiple emulsions (W/O/W or O/W/O), microemulsions, vesicular dispersions of ionic and/or nonionic type, or wax/aqueous phase dispersions. It may also be in the form of hot-cast sticks or loose or compacted powders.

These compositions are prepared according to the usual methods.

According to a specific embodiment, the composition according to the invention is provided in the form of an anhydrous composition.

According to another particular embodiment, the composition according to the invention is in the form of an aqueous composition, in particular in the form of a water-in-oil emulsion comprising a continuous oily phase and an aqueous phase dispersed in the said oily phase, or in the form of an oil-in-water emulsion comprising a continuous aqueous phase and an oily phase dispersed in the said aqueous phase.

For the purposes of the present invention, the term “anhydrous” is understood to mean a composition comprising a content of less than or equal to 1% by weight of water and preferably of less than or equal to 0.5% by weight, relative to the total weight of the said composition, or even being free of water. If appropriate, such small amounts of water may in particular be introduced by ingredients of the composition, which may comprise residual amounts thereof.

When the composition is in the form of an anhydrous composition, the proportion of the fatty phase may range, for example, from 30% to 99% by weight and preferably from 50% to 90% by weight relative to the total weight of the composition.

When the composition is in the form of an emulsion, the proportion of the fatty phase may range, for example, from 1% to 80% by weight and preferably from 5% to 40% by weight relative to the total weight of the composition.

This indicated amount does not comprise the content of lipophilic surfactants.

The composition according to the invention may comprise at least one additional fatty substance other than the silanes of formula (I) and/or oligomers thereof.

For the purpose of the present invention, the term “fatty substance” is intended to mean an organic compound which is insoluble in water at normal temperature (25° C.) and at atmospheric pressure (760 mmHg, i.e. 1.013×10⁵ Pa), that is to say with a solubility of less than 4% by weight, preferably of less than 1% by weight and even more preferentially of less than 0.1% by weight. They have in their structure at least one hydrocarbon-based chain comprising at least 6 carbon atoms or a sequence of at least two siloxane groups. In addition, the fatty substances are soluble in organic solvents under the same temperature and pressure conditions, for instance chloroform, ethanol or benzene.

The fatty substances are not etherified with oxyalkylenated or glycerolated groups.

The fatty substances of the invention may be liquid or non-liquid at room temperature (25° C.) and at atmospheric pressure (760 mmHg; i.e. 1.013×10⁵ Pa).

The liquid fatty substances of the invention preferably have a viscosity of less than or equal to 2 Pa·s, better still less than or equal to 1 Pa·s and even better still less than or equal to 0.1 Pa·s at a temperature of 25° C. and at a shear rate of 1 s⁻¹.

Liquid fatty substances that may be mentioned include silicone or non-silicone oils.

The term “non-silicone oil” means an oil not containing any silicon atoms (Si) and the term “silicone oil” means an oil containing at least one silicon atom.

More particularly, the fatty substances are chosen from C₆-C₁₆ hydrocarbons, hydrocarbons containing more than 16 carbon atoms, non-silicone oils of animal origin, plant oils of triglyceride type, synthetic triglycerides, fluoro oils, fatty alcohols, esters of fatty acid and/or of fatty alcohol other than triglycerides, and plant waxes, non-silicone waxes and silicones, and mixtures thereof.

It is recalled that the fatty alcohols, esters and acids more particularly have at least one linear or branched, saturated or unsaturated hydrocarbon-based group comprising 6 to 30 and better still from 8 to 30 carbon atoms, which is optionally substituted, in particular with one or more hydroxyl groups (in particular 1 to 4). If they are unsaturated, these compounds may comprise one to three conjugated or unconjugated carbon-carbon double bonds.

As regards the C₆-C₁₆ hydrocarbons, they are linear or branched, and possibly cyclic, and are preferably alkanes. Examples that may be mentioned include hexane, dodecane and isoparaffins such as isohexadecane and isodecane.

A hydrocarbon-based oil of animal origin that may be mentioned is perhydrosqualene.

The triglyceride oils of plant or synthetic origin are preferably chosen from liquid fatty acid triglycerides comprising from 6 to 30 carbon atoms, for instance heptanoic or octanoic acid triglycerides, or alternatively, for example, sunflower oil, corn oil, soybean oil, marrow oil, grapeseed oil, sesame seed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil.

The linear or branched hydrocarbons of mineral or synthetic origin having more than 16 carbon atoms are preferably chosen from liquid paraffins, petrolatum, liquid petrolatum, polydecenes or hydrogenated polyisobutene, such as Parleam®.

As regards the C₆-C₁₆ alkanes, they are linear or branched, and optionally cyclic. Examples that may be mentioned include hexane, dodecane and isoparaffins such as isohexadecane and isodecane.

As oils of animal, plant, mineral or synthetic origin that may be used in the composition of the invention, examples that may be mentioned include:

the fluoro oils may be chosen from perfluoromethylcyclopentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxybutane and nonafluoroethoxyisobutane; perfluoromorpholine derivatives such as 4-trifluoromethylperfluoromorpholine sold under the name PF 5052® by the company 3M.

The fatty alcohols which are suitable for the implementation of the invention are more particularly chosen from saturated or unsaturated and linear or branched alcohols comprising from 6 to 30 carbon atoms and preferably from 8 to 30 carbon atoms. Mention may be made, for example, of cetyl alcohol, stearyl alcohol and a mixture thereof (cetylstearyl alcohol), octyldodecanol, 2-butyloctanol, 2-hexyldecanol, 2-undecylpentadecanol, oleyl alcohol or linoleyl alcohol.

As regards the esters of fatty acid and/or of fatty alcohols, advantageously other than the triglycerides mentioned previously, mention may be made especially of esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being greater than or equal to 6 and more advantageously greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononanoate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still within the context of this variant, it is also possible to use esters of C₄-C₂₂ dicarboxylic or tricarboxylic acids and of C₁-C₂₂ alcohols, and esters of monocarboxylic, dicarboxylic or tricarboxylic acids and of C₂-C₂₆ di-, tri-, tetra- or penta-hydroxy alcohols.

Mention may in particular be made of: diethyl sebacate; diisopropyl sebacate; diisopropyl adipate; di(n-propyl) adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; and polyethylene glycol distearates.

Among the esters mentioned above, use is preferably made of ethyl, isopropyl, myristyl, cetyl or stearyl palmitate, 2-ethylhexyl palmitate, 2-octyldecyl palmitate, alkyl myristates, such as isopropyl, butyl, cetyl or 2-octyldodecyl myristate, hexyl stearate, butyl stearate, isobutyl stearate, dioctyl malate, hexyl laurate, 2-hexyldecyl laurate, isononyl isononanoate or cetyl octanoate.

The composition may also comprise, as fatty ester, sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Mention may be made, as suitable sugars, for example, of sucrose (or saccharose), glucose, galactose, ribose, fucose, maltose, fructose, mannose, arabinose, xylose and lactose, and derivatives thereof, in particular alkyl derivatives, such as methyl derivatives, for instance methylglucose.

The sugar esters of fatty acids may be selected especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise from one to three conjugated or unconjugated carbon-carbon double bonds.

The esters according to this alternative form may also be chosen from mono-, di-, tri- and tetraesters, polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters.

More particularly, use is made of monoesters and diesters and in particular mono- or di-oleate, -stearate, -behenate, -oleate/palmitate, -linoleate, -linolenate or -oleate/stearate of sucrose, of glucose or of methylglucose.

An example that may be mentioned is the product sold under the name Glucate® DO by the company Amerchol, which is a methylglucose dioleate.

Examples of esters or mixtures of esters of sugar and of fatty acid that may also be mentioned include:

the products sold under the names F160, F140, F110, F90, F70 and SL40 by the company Crodesta, respectively denoting sucrose palmitostearates formed from 73% monoester and 27% diester and triester, from 61% monoester and 39% diester, triester and tetraester, from 52% monoester and 48% diester, triester and tetraester, from 45% monoester and 55% diester, triester and tetraester, from 39% monoester and 61% diester, triester and tetraester, and sucrose monolaurate;

the products sold under the name Ryoto Sugar Esters, for example referenced B370 and corresponding to sucrose behenate formed from 20% monoester and 80% diester-triester-polyester;

the sucrose monopalmitate/stearate-dipalmitate/stearate sold by the company Goldschmidt under the name Tegosoft® PSE.

The non-silicone wax(es) are chosen in particular from carnauba wax, candelilla wax, esparto wax, paraffin wax, ozokerite, plant waxes, such as olive tree wax, rice wax, hydrogenated jojoba wax or absolute flower waxes, such as the blackcurrant blossom essential wax sold by Bertin (France), or animal waxes, such as beeswaxes or modified beeswaxes (cerabellina); other waxes or waxy starting materials which can be used according to the invention are in particular marine waxes, such as that sold by Sophim under the reference M82, polyethylene waxes or polyolefin waxes in general.

The silicones that may be used in the cosmetic compositions of the present invention are volatile or nonvolatile, cyclic, linear or branched silicones, which are unmodified or modified with organic groups, having a viscosity from 5×10⁻⁶ to 2.5 m²/s at 25° C., and preferably 1×10⁻⁵ to 1 m²/s.

The silicones that may be used in accordance with the invention may be in the form of oils, waxes, resins or gums.

More particularly, the fatty substances are chosen from compounds that are liquid or pasty at room temperature (25° C.) and at atmospheric pressure.

Preferably, the fatty substance is a compound that is liquid at a temperature of 25° C. and at atmospheric pressure.

The fatty substances are advantageously chosen from C₆-C₁₆ alkanes, nonsilicone oils of plant, mineral or synthetic origin, fatty alcohols, and esters of fatty acid and/or of fatty alcohol, or mixtures thereof. Preferably, the fatty substance is chosen from liquid petrolatum, C₆-C₁₆ alkanes, polydecenes, liquid esters of fatty acid and/or of fatty alcohol, and liquid fatty alcohols, or mixtures thereof.

When they are present, the fatty substances may represent from 0.01% to 95%, preferably from 0.1% to 90% by weight, better still from 0.5% to 80% by weight and even better still from 1% to 30% by weight relative to the total weight of the composition.

The composition according to the invention may also comprise at least one surfactant.

The surfactants are chosen from anionic, cationic, nonionic, amphoteric and zwitterionic surfactants.

The term “anionic surfactant” means a surfactant comprising, as ionic or ionizable groups, only anionic groups. These anionic groups are preferably chosen from the groups —C(O)OH, —C(O)O—, —SO₃H, —S(O)₂O—, —OS(O)₂OH, —OS(O)₂O—, —P(O)OH₂, —P(O)₂O—, —P(O)C₂—, —P(OH)₂, ═P(O)OH, —P(OH)O—, ═P(O)O—, ═POH, ═PO—, the anionic parts comprising a cationic counterion such as an alkali metal, an alkaline-earth metal or an ammonium.

As examples of anionic surfactants that may be used in the composition according to the invention, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, α-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N-acyltaurates, salts of alkyl monoesters of polyglycoside-polycarboxylic acids, acyllactylates, D-galactoside uronic acid salts, alkyl ether carboxylic acid salts, alkylaryl ether carboxylic acid salts, alkylamido ether carboxylic acid salts; and the corresponding non-salified forms of all these compounds; the alkyl and acyl groups of all these compounds comprising from 6 to 40 carbon atoms and the aryl group denoting a phenyl group.

These compounds can be oxyethylenated and then preferably comprise from 1 to 50 ethylene oxide units.

The salts of C₆-C₂₄ alkyl monoesters of polyglycoside-polycarboxylic acids can be selected from C₆-C₂₄ alkyl polyglycoside-citrates, C₆-C₂₄ alkyl polyglycoside-tartrates and C₆-C₂₄ alkyl polyglycoside-sulfosuccinates.

When the anionic surfactant(s) are in salt form, they may be chosen from alkali metal salts such as the sodium or potassium salt and preferably the sodium salt, ammonium salts, amine salts and in particular amino alcohol salts or alkaline-earth metal salts such as the magnesium salts.

Examples of amino alcohol salts that may especially be mentioned include monoethanolamine, diethanolamine and triethanolamine salts, monoisopropanolamine, diisopropanolamine or triisopropanolamine salts, 2-amino-2-methyl-1-propanol salts, 2-amino-2-methyl-1,3-propanediol salts and tris(hydroxymethyl)aminomethane salts.

Alkali metal or alkaline-earth metal salts, and in particular sodium or magnesium salts, are preferably used.

Among the anionic surfactants mentioned, use is preferably made of (C₆-C₂₄)alkyl sulfates, (C₆-C₂₄)alkyl ether sulfates comprising from 2 to 50 ethylene oxide units, especially in the form of alkali metal, ammonium, amino alcohol and alkaline-earth metal salts, or a mixture of these compounds.

In particular, use is preferably made of (C₁₂-C₂₀)alkyl sulfates, (C₁₂-C₂₀)alkyl ether sulfates comprising from 2 to 20 ethylene oxide units, especially in the form of alkali metal, ammonium, amino alcohol and alkaline-earth metal salts, or a mixture of these compounds. Better still, it is preferred to use sodium lauryl ether sulfate containing 2.2 mol of ethylene oxide.

Examples of additional nonionic surfactants that may be used in the compositions of the present invention are described, for example, in the Handbook of Surfactants by M. R. Porter, published by Blackie & Son (Glasgow and London), 1991, pp 116-178. They are especially chosen from alcohols, α-diols and (C₁₋₂₀)alkylphenols, these compounds being etherified with ethoxylated, propoxylated or glycerolated groups and containing at least one fatty chain comprising, for example, from 8 to 18 carbon atoms, the number of ethylene oxide or propylene oxide groups possibly ranging especially from 2 to 50, and the number of glycerol groups possibly ranging especially from 2 to 30.

Mention may also be made of condensates of ethylene oxide and of propylene oxide with fatty alcohols; polyethoxylated fatty amides preferably having from 2 to 30 ethylene oxide units, polyglycerolated fatty amides containing on average from 1 to 5, and in particular from 1.5 to 4, glycerol groups, ethoxylated fatty acid esters of sorbitan containing from 2 to 30 ethylene oxide units, fatty acid esters of sucrose, fatty acid esters of polyethylene glycol, (C₆₋₂₄ alkyl)polyglycosides, N—(C₆₋₂₄ alkyl)glucamine derivatives, amine oxides such as (C₁₀₋₁₄alkyl)amine oxides or N(C₁₀₋₁₄ acyl)aminopropylmorpholine oxides.

The amphoteric or zwitterionic surfactants that may be used in the present invention may especially be secondary or tertiary aliphatic amine derivatives in which the aliphatic group is a linear or branched chain comprising from 8 to 22 carbon atoms and the said amine derivatives containing at least one anionic group, for instance a carboxylate, sulfonate, sulfate, phosphate or phosphonate group. Mention may be made in particular of (C₈₋₂₀)alkylbetaines, sulfobetaines, (C₈₋₂₀)alkylamido(C₆₋₈)alkylbetaines or (C₈₋₂₀)alkylamido(C₆₋₈)alkylsulfobetaines.

Among the optionally quaternized secondary or tertiary aliphatic amine derivatives that may be used, as defined above, mention may also be made of the compounds of respective structures (B1) and (B2) below:

Ra—C(O)—NH—CH₂—CH₂—N+(Rb)(Rc)-CH₂C(O)O—, M+, X— (B1)

in which formula:
Ra represents a C₁₀-C₃₀ alkyl or alkenyl group derived from an acid Ra—COON preferably present in hydrolysed coconut oil, or a heptyl, nonyl or undecyl group;
Rb represents a p-hydroxyethyl group; and
Rc represents a carboxymethyl group;
M+ represents a cationic counterion derived from an alkali metal or alkaline-earth metal, such as sodium, an ammonium ion or an ion derived from an organic amine; and
X— represents an organic or inorganic anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate; or alternatively M+ and X— are absent;

Ra′—C(O)—NH—CH₂—CH₂—N(B)(B′) (B2)

in which formula:
B represents the group —CH₂—CH₂—O—X′;
B′ represents the group —(CH₂)zY′, with z=1 or 2;
X′ represents the group —CH₂—C(O)OH, —CH₂—C(O)OZ′, —CH₂—CH₂—C(O)OH or —CH₂—CH₂—C(O)OZ′, or a hydrogen atom;
Y′ represents the group —C(O)OH, —C(O)OZ′, —CH₂—CH(OH)—SO₃H or the group —CH₂—CH(OH)—SC₃—Z′;
Z′ represents a cationic counterion derived from an alkali metal or alkaline-earth metal, such as sodium, an ammonium ion or an ion derived from an organic amine;
Ra′ represents a C₁₀-C₃₀ alkyl or alkenyl group of an acid Ra′—C(O)OH preferably present in coconut oil or in hydrolysed linseed oil, an alkyl group, especially of C₁₇ and its iso form, or an unsaturated C₁₇ group.

These compounds of formula (B1) or (B2) are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Use may also be made of compounds of formula (B′2):

Ra″—NH—CH(Y″)—(CH₂)n-C(O)—NH—(CH₂)n′-N(Rd)(Re) (B′2)

in which formula:
Y″ represents the group —C(O)OH, —C(O)OZ″, —CH₂—CH(OH)—SO₃H or the group —CH₂—CH(OH)—SC₃—Z″;
Rd and Re, independently of each other, represent a C₁-C₄ alkyl or hydroxyalkyl radical;
Z″ represents a cationic counterion derived from an alkali metal or alkaline-earth metal, such as sodium, an ammonium ion or an ion derived from an organic amine;
Ra″ represents a C₁₀-C₃₀ alkyl or alkenyl group of an acid Ra′—C(O)OH preferably present in coconut oil or in hydrolysed linseed oil.
n and n′ denote, independently of each other, an integer ranging from 1 to 3.

Among the compounds of formula (B′2), mention may be made of the compound classified in the CTFA dictionary under the name sodium diethylaminopropyl cocoaspartamide and sold by the company Chimex under the name Chimexane HB.

Among the amphoteric or zwitterionic surfactants mentioned above, use is preferably made of (C₈-C₂₀)alkylbetaines such as cocobetaine, (C₈-C₂₀)alkylamido(C₃-C₈)alkylbetaines such as cocoamidopropylbetaine, and the compounds of formula (B′2) such as the sodium salt of diethylaminopropyl laurylaminosuccinamate (INCI name: sodium diethylaminopropyl cocoaspartamide), and mixtures thereof. More preferentially, the amphoteric or zwitterionic surfactant(s) are chosen from cocamidopropylbetaine and cocobetaine.

The cationic surfactant(s) which can be used in the composition according to the invention comprise, for example, salts of optionally polyoxyalkylenated primary, secondary or tertiary fatty amines, quaternary ammonium salts, and mixtures thereof.

Examples of quaternary ammonium salts that may especially be mentioned include:

those corresponding to the general formula (C1) below:

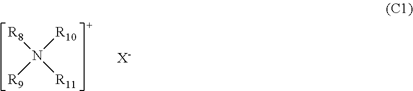

in which formula:
R₈ to R₁₁, which may be identical or different, represent a linear or branched aliphatic group comprising from 1 to 30 carbon atoms, or an aromatic group such as aryl or alkylaryl, it being understood that at least one of the groups R₈ to R₁₁ comprises from 8 to 30 carbon atoms and preferably from 12 to 24 carbon atoms; and
X⁻ represents an organic or inorganic anionic counterion, such as that chosen from halides, acetates, phosphates, nitrates, (C₁-C₄)alkyl sulfates, (C₁-C₄)alkylsulfonates or (C₁-C₄)alkylarylsulfonates, in particular methyl sulfate and ethyl sulfate.
   The aliphatic groups of R₈ to R₁₁ may also comprise heteroatoms such as in particular oxygen, nitrogen, sulfur and halogens.

The aliphatic groups of R₈ to R₁₁ are chosen, for example, from C₁-C₃₀ alkyl, C₁-C₃₀ alkoxy, polyoxy(C₂-C₆)alkylene, C₁-C₃₀ alkylamide, (C₁₂-C₂₂)alkylamido(C₂-C₆)alkyl, (C₁₂-C₂₂)alkylacetate and C₁-C₃₀ hydroxyalkyl groups, and X⁻ is an anionic counterion chosen from halides, phosphates, acetates, lactates, (C₁-C₄)alkyl sulfates, and (C₁-C₄)alkylsulfonates or (C₁-C₄)alkylarylsulfonates.

Among the quaternary ammonium salts of formula (C1), preference is given firstly to tetraalkylammonium chlorides, for instance dialkyldimethyl-ammonium or alkyltrimethylammonium chlorides in which the alkyl group comprises approximately from 12 to 22 carbon atoms, in particular behenyltrimethylammonium chloride, distearyldimethylammonium chloride, cetyltrimethylammonium chloride, benzyldimethylstearylammonium chloride, or else, secondly, distearoylethylhydroxyethylmethylammonium methosulfate, dipalmitoylethylhydroxyethylammonium methosulfate or distearoylethylhydroxyethylammonium methosulfate, or else, lastly, palmitylamidopropyltrimethylammonium chloride or stearamidopropyldimethyl(myristyl acetate)ammonium chloride, sold under the name Ceraphyl® 70 by the company Van Dyk;

quaternary ammonium salts of imidazoline, for instance those of formula (C2) below:

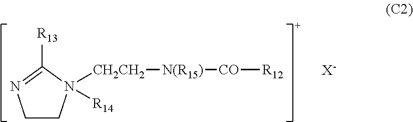

in which formula:
R₁₂ represents an alkenyl or alkyl group comprising from 8 to 30 carbon atoms, for example tallow fatty acid derivatives;
R₁₃ represents a hydrogen atom, a C₁-C₄ alkyl group or an alkenyl or alkyl group comprising from 8 to 30 carbon atoms;
R₁₄ represents a C₁-C₄ alkyl group;
R₁₅ represents a hydrogen atom or a C₁-C₄ alkyl group;
X⁻ represents an organic or inorganic anionic counterion, such as that chosen from halides, phosphates, acetates, lactates, (C₁-C₄)alkyl sulfates, (C₁-C₄)alkylsulfonates or (C₁-C₄)alkylarylsulfonates.
R₁₂ and R₁₃ preferably denote a mixture of alkyl or alkenyl groups comprising from 12 to 21 carbon atoms, for example tallow fatty acid derivatives, R₁₄ denotes a methyl group, and R₁₅ denotes a hydrogen atom. Such a product is sold, for example, under the name Rewoquat® W 75 by the company Rewo;

quaternary diammonium or triammonium salts, particularly of formula (C3) below:

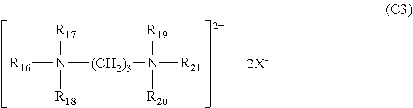

in which formula:
R₁₆ denotes an alkyl group comprising approximately from 16 to 30 carbon atoms, which is optionally hydroxylated and/or interrupted with one or more oxygen atoms;
R₁₇ is chosen from hydrogen, an alkyl group comprising from 1 to 4 carbon atoms or a group —(CH₂)₃—N*(R₁₆ₐ)(R₁₇ₐ)(R₁₈ₐ), X⁻;
R₁₆ₐ, R₁₇ₐ, R₁₈ₐ, R₁₈, R₁₉, R₂₀ and R₂₁, which may be identical or different, are chosen from hydrogen and an alkyl group comprising from 1 to 4 carbon atoms; and
X⁻, which may be identical or different, represent an organic or inorganic anionic counterion, such as that chosen from halides, acetates, phosphates, nitrates, (C₁-C₄)alkyl sulfates, (C₁-C₄)alkyl- or (C₁-C₄)alkylarylsulfonates, in particular methyl sulfate and ethyl sulfate.

Such compounds are, for example, Finquat CT-P, provided by Finetex (Quaternium 89), or Finquat CT, provided by Finetex (Quaternium 75);

quaternary ammonium salts containing one or more ester functions, such as those of formula (C4) below:

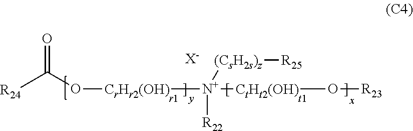

in which formula:
R₂₂ is chosen from C₁-C₆ alkyl and C₁-C₆ hydroxyalkyl or dihydroxyalkyl groups,
R₂₃ is chosen from:
   the group

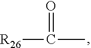

linear or branched, saturated or unsaturated C₁-C₂₂ hydrocarbon-based groups R₂₇,
a hydrogen atom,
R₂₅ is selected from:
   the group

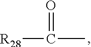

the groups R₂₉, which are linear or branched, saturated or unsaturated C₁-C₆ hydrocarbon-based groups;
a hydrogen atom,
R₂₄, R₂₆ and R₂₈, which may be identical or different, are chosen from linear or branched, saturated or unsaturated C₇-C₂₁ hydrocarbon-based groups;
r, s and t, which may be identical or different, are integers having values from 2 to 6,
r1 and t1, which may be identical or different, are equal to 0 or 1, with r2+r1=2r and t1+t2=2t,
y is an integer ranging from 1 to 10,
x and z, which may be identical or different, are integers ranging from 0 to 10,
X⁻ represents an organic or inorganic anionic counterion,
   with the proviso that the sum x+y+z is from 1 to 15, that when x is 0, then R₂₃ denotes R₂₇ and that when z is 0, then R₂₅ denotes R₂₉.

The alkyl groups R₂₂ may be linear or branched, and more particularly linear.

Preferably, R₂₂ denotes a methyl, ethyl, hydroxyethyl or dihydroxypropyl group, and more particularly a methyl or ethyl group.

Advantageously, the sum x+y+z is from 1 to 10.

When R₂₃ is a hydrocarbon-based group R₂₇, it may be long and may have from 12 to 22 carbon atoms, or may be short and may have from 1 to 3 carbon atoms.

When R₂₅ is a hydrocarbon-based group R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based groups, and more particularly from linear or branched, saturated or unsaturated C₁₁-C₂₁ alkyl and alkenyl groups.

Preferably, x and z, which may be identical or different, are equal to 0 or 1.

y is advantageously equal to 1.

Preferably, r, s and t, which may be identical or different, equal 2 or 3, and even more particularly are equal to 2.

The anionic counterion k is preferably a halide, such as chloride, bromide or iodide; a (C₁-C₄)alkyl sulfate or a (C₁-C₄)alkylsulfonate or (C₁-C₄)alkylarylsulfonate. However, it is possible to use methanesulfonate, phosphate, nitrate, tosylate, an anion derived from an organic acid, such as acetate or lactate, or any other anion that is compatible with the ammonium containing an ester function.

The anionic counterion k is even more particularly chloride, methyl sulfate or ethyl sulfate.

Use is made more particularly, in the composition according to the invention, of the ammonium salts of formula (C4) in which:
R₂₂ denotes a methyl or ethyl group,
x and y are equal to 1,
z is equal to 0 or 1,
r, s and t are equal to 2,
R₂₃ is chosen from:
   the group

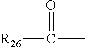

methyl, ethyl or C₁₄-C₂₂ hydrocarbon-based groups,
a hydrogen atom,
R₂₅ is chosen from:
   the group

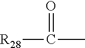

a hydrogen atom,
R₂₄, R₂₆ and R₂₈, which may be identical or different, are chosen from linear or branched, saturated or unsaturated C₁₃-C₁₇ hydrocarbon-based groups, and preferably from linear or branched, saturated or unsaturated C₁₃-C₁₇ alkyl and alkenyl groups.

Advantageously, the hydrocarbon-based radicals are linear.

Among the compounds of formula (C4), examples that may be mentioned include salts, especially the chloride or methyl sulfate, of diacyloxyethyldimethylammonium, diacyloxyethylhydroxyethylmethylammonium, monoacyloxyethyldihydroxyethylmethylammonium, triacyloxyethylmethylammonium or monoacyloxyethylhydroxyethyldimethylammonium, and mixtures thereof. The acyl groups preferably contain 14 to 18 carbon atoms and are obtained more particularly from a plant oil, such as palm oil or sunflower oil. When the compound contains several acyl groups, these groups may be identical or different.

These products are obtained, for example, by direct esterification of triethanolamine, triisopropanolamine, an alkyldiethanolamine or an alkyldiisopropanolamine, which are optionally oxyalkylenated, with fatty acids or with fatty acid mixtures of plant or animal origin, or by transesterification of the methyl esters thereof. This esterification is followed by a quaternization by means of an alkylating agent such as an alkyl halide, preferably methyl or ethyl halide, a dialkyl sulfate, preferably dimethyl or diethyl sulfate, methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company Ceca or Rewoquat® WE 18 by the company Rewo-Witco.

The composition according to the invention may contain, for example, a mixture of quaternary ammonium salts of monoesters, diesters and triesters with a weight majority of diester salts.

It is also possible to use the ammonium salts containing at least one ester function that are described in U.S. Pat. No. 4,874,554 and U.S. Pat. No. 4,137,180.

Use may be made of behenoylhydroxypropyltrimethylammonium chloride sold by KAO under the name Quatarmin BTC 131.

Preferably, the ammonium salts containing at least one ester function contain two ester functions.

Among the cationic surfactants that may be present in the composition according to the invention, it is more particularly preferred to choose cetyltrimethylammonium, behenyltrimethylammonium and dipalmitoylethyl-hydroxyethylmethylammonium salts, and mixtures thereof, and more particularly behenyltrimethylammonium chloride, cetyltrimethylammonium chloride, and dipalmitoylethylhydroxyethylammonium methosulfate, and mixtures thereof.

When they are present, the surfactants may represent from 0.01% to 30%, preferably from 0.1% to 20% by weight and better still from 1% to 10% by weight, relative to the total weight of the composition.

In the compositions for the skin, the surfactants are generally present in the composition in a proportion ranging from 0.1% to 30% by weight and preferably from 0.2% to 20% by weight relative to the total weight of the composition and are more particularly chosen from the following:

For the W/O emulsions, examples of emulsifiers that may be mentioned include dimethicone copolyols such as the mixture of cyclomethicone and of dimethicone copolyol sold under the name DC 5225 C by the company Dow Corning, and alkyl dimethicone copolyols such as the laurylmethicone copolyol sold under the name Dow Corning 5200 Formulation Aid by the company Dow Corning and the cetyl dimethicone copolyol sold under the name Abil EM 90® by the company Goldschmidt, or the mixture polyglyceryl-4 isostearate/cetyl dimethicone copolyol/hexyl laurate sold under the name Abil WE 09 by the company Goldschmidt. One or more co-emulsifiers may also be added thereto. The coemulsifier can advantageously be chosen from the group consisting of polyol alkyl esters. Mention may in particular be made, as polyol alkyl esters, of glycerol and/or sorbitan esters, for example polyglyceryl isostearate, such as the product sold under the name Isolan GI 34 by Goldschmidt, sorbitan isostearate, such as the product sold under the name Arlacel 987 by ICI, sorbitan glyceryl isostearate, such as the product sold under the name Arlacel 986 by ICI, and mixtures thereof.

Mention may be made, for the O/W emulsions, for example, as emulsifiers, of nonionic surfactants and in particular esters of polyols and of fatty acid having a saturated or unsaturated chain comprising, for example, from 8 to 24 carbon atoms and better still from 12 to 22 carbon atoms, and their oxyalkylenated derivatives, that is to say derivatives comprising oxyethylene and/or oxypropylene units, such as glyceryl esters of C₈-C₂₄ fatty acid, and their oxyalkylenated derivatives; polyethylene glycol esters of C₈-C₂₄ fatty acid, and their oxyalkylenated derivatives; sorbitol esters of C₈-C₂₄ fatty acid, and their oxyalkylenated derivatives; fatty alcohol ethers; sugar ethers of C₈-C₂₄ fatty alcohols, and mixtures thereof.

Mention may in particular be made, as glyceryl ester of fatty acid, of glyceryl stearate (glyceryl mono-, di- and/or tristearate) (CTFA name: glyceryl stearate) or glyceryl ricinoleate, and mixtures thereof.

Mention may in particular be made, as polyethylene glycol ester of fatty acid, of polyethylene glycol stearate (polyethylene glycol mono-, di- and/or tristearate) and more especially polyethylene glycol 50 OE monostearate (CTFA name: PEG-50 stearate), polyethylene glycol 100 OE monostearate (CTFA name: PEG-100 stearate) and mixtures thereof.

Use may also be made of mixtures of these surfactants, such as, for example, the product containing glyceryl stearate and PEG-100 stearate, sold under the name Arlacel 165 by Uniqema, and the product containing glyceryl stearate (glyceryl mono/distearate) and potassium stearate, sold under the name Tegin by Goldschmidt (CTFA name: glyceryl stearate SE).

Mention may be made, as fatty alcohol ethers, for example, of polyethylene glycol ethers of fatty alcohol comprising from 8 to 30 carbon atoms and in particular from 10 to 22 carbon atoms, such as polyethylene glycol ethers of cetyl alcohol, stearyl alcohol or cetearyl alcohol (mixture of cetyl alcohol and stearyl alcohol). Mention may be made, for example, of ethers comprising from 1 to 200 and preferably from 2 to 100 oxyethylene groups, such as those with the CTFA name Ceteareth-20 or Ceteareth-30, and mixtures thereof.

Mention may be made, as examples of sugar mono- or polyalkyl esters or ethers, of methyl glucose isostearate, sold under the name Isolan-IS by Degussa Goldschmidt, or else sucrose distearate, sold under the name Crodesta F50 by Croda, and sucrose stearate, sold under the name Ryoto sugar ester S 1570 by Mitsubishi Kagaku Foods.

Mention may also be made of lipoamino acids and their salts, such as monosodium and disodium acylglutamates, such as, for example, monosodium stearoyl glutamate, sold under the name Amisoft HS-11PF, and disodium stearoyl glutamate, sold under the name Amisoft HS-21P, by Ajinomoto.

The composition according to the invention may comprise an aqueous phase, the amount of which may range, for example, from 10% to 98% by weight, preferably from 30% to 98% by weight, better still from 40% to 98% by weight and even better still from 50% to 95% by weight relative to the total weight of the composition.

Conventionally, the aqueous phase may contain, besides water, one or more water-soluble solvents chosen from polyols (or polyhydric alcohols) and water-soluble lower alcohol(s), and mixtures thereof. The term “lower alcohol” means an alcohol comprising from 1 to 6 and preferably from 1 to 4 carbon atoms. Examples of lower alcohols that may be mentioned include ethanol, isopropanol and butanol, and mixtures thereof.

Examples of polyols that may be mentioned include glycerol; glycols such as propylene glycol, 1,3-butylene glycol, dipropylene glycol or butylene glycol; sorbitol; sugars such as glucose, fructose, maltose, lactose and sucrose; and mixtures thereof.

The amount of water-soluble solvents (polyols and lower alcohols) may range, for example, from 0.5% to 30% by weight, preferably from 0.5% to 20% by weight and better still from 1% to 15% by weight relative to the total weight of the composition.

In the present invention, the term “water-soluble solvent” denotes a compound that is liquid at room temperature and water-miscible (miscibility in water of greater than 50% by weight at 25° C. and atmospheric pressure).

### Adjuvants

In a known manner, the composition of the invention may also contain adjuvants that are common in cosmetics and/or dermatology other than lipophilic thickeners, such as preserving agents, antioxidants, complexing agents, pH regulators (acidic or basic), fragrances, fillers, bactericides, odour absorbers, dyestuffs (pigments and dyes), film-forming polymers including cationic polymers, silicone or non-silicone sunscreens, plasticizers, antifoams, silanes other than those of formula (I), antidandruff agents, anti-seborrhoeic agents or anti-grease agents, agents for combating hair loss and/or for promoting hair regrowth, penetrants, ceramides and pseudoceramides, vitamins and provitamins including panthenol, nacreous agents or opacifiers, skin bleaching agents, antiwrinkle agents, anti-desquamating agents, moisturizers, antiageing agents, self-tanning agents, water-soluble thickeners, in particular polyacrylamides, acrylic homopolymers and copolymers, and acrylamidomethylpropanesulfonic acid homopolymers and copolymers, and also lipid vesicles. The amounts of these various adjuvants are those conventionally used in the field under consideration, for example from 0.01% to 20% of the total weight of the composition. Depending on their nature, these adjuvants may be introduced into the fatty phase, into the aqueous phase and/or into the lipid vesicles.

Needless to say, a person skilled in the art will take care to select this or these optional additional compound(s), and/or the amount thereof, such that the mattifying/soft focus properties of the composition according to the invention are not, or are not substantially, adversely affected by the envisaged addition. In the patent application, unless specifically mentioned otherwise, the contents are expressed on a weight basis relative to the total weight of the composition.

The compositions of the invention may be used in any cosmetic or dermatological application, for example in cosmetics for caring for the skin, the hair, the scalp, the eyelashes, the eyebrows, the nails or mucous membranes (the lips), for example as protecting, treating or care products for the face, the hands or the body, as skin-cleansing products (for the face or the body), as makeup products (for example foundations) or as haircare products.

In the case of a hair treatment, the application of the composition according to the invention to the hair may or may not be followed by rinsing.

When the application of the composition is followed by rinsing, the leave-on time of the composition on the keratin materials ranges from a few seconds to 60 minutes, better still from 5 seconds to 30 minutes, even better still from 10 seconds to 10 minutes.

Whether in rinsed mode or non-rinsed mode, the application of the composition may take place in the presence of heat. The heating device may be a hairdryer, a hood dryer, a round iron or a flat iron. The heating temperature may be between 40 and 220° C., especially about 200° C. when a straightening iron is used.

The application of the composition according to the invention to the hair may take place on dry hair or on wet hair. It may in particular be carried out after a shampooing operation or after a pretreatment at acidic or basic pH.

The application of the composition of the invention may also be followed by a post-treatment at acidic or basic pH, followed especially by drying at room temperature or by drying by supplying heat, using a hairdryer, a hood or a round or flat iron.

The composition of the invention may be applied to the hair in combination with chemical treatments such as oxidations, reductions, buffer solutions or mechanical treatments.

The examples that follow are aimed at illustrating the compositions and processes according to this invention, but are not in any way a limitation of the scope of the invention. All the parts and percentages in the examples are given on a weight basis and all the measurements were obtained at about 25° C., unless otherwise mentioned.

### EXAMPLE 1

The moisturizing cream having the following compositions was prepared:

| | | | B |
|---|---|---|---|
| | | A | (invention) |
| A1 | Glyceryl stearate (and) PEG-100 stearate | 3 | 3 |
| | (Arlacel 165 from Croda) | | |
| | Hexyldecyl laurate (and) hexyldecanol | 4 | 4 |
| | (Cetiol PGL) | | |
| | Cyclohexamethicone | 8 | — |
| | Poly C₁₀₋₃₀ alkyl acrylate (Intelimer | 0.5 | 0.5 |
| | IPA 13-1) | | |
| | Dimethicone (and) dimethiconol | 2 | — |
| | Octyltriethoxysilane | — | 10 |
| B | Glycerol | 15 | 15 |
| | Preserving agents | qs | qs |
| | Water | qs | qs |
| | | for 100 | for 100 |
| | Xanthan gum | 0.15 | 0.15 |
| C | Ammonium polyacryloyldimethyl taurate | 0.5 | 0.5 |
| | (Hostacerin AMPS ® from Clariant) | | |
| D | Aluminium starch octenylsuccinate | 3 | 3 |
| | (Dry Flo Plus) | | |

Procedure:

Heat phase A to 75° C., preferably on a water bath, and stir from time to time to homogenize. Heat phase B to 80° C. and then cool to 75° C. Form the emulsion conventionally using a mixer. Mix intermittently up to 60° C. and then stir using a Rayneri blender. Add phase C and continue stirring with the Rayneri blender. At 40° C., add D and then leave to cool to room temperature with stirring.

### EVALUATION—COMPARATIVE EXAMPLES

Compositions A and B (according to the invention) were evaluated by a panel of 10 experts. The comparative composition A contains standard silicone oils in place of the silane.

For each of the compositions, the glidance and the sheen (shiny effect) on application and after penetration into the skin are evaluated according to the following protocol.

The glidance on application was evaluated, monadically, by a panel of experts trained in the description of care products.

The samples are presented in random order to each panellist. Ten experts evaluated the glidance in the following manner: on a hand precleaned with water and liquid soap and wiped dry with a tissue, 0.05 ml of product was applied to the top half of the hand (five passes with the index and middle fingers). The product was evaluated during the five passes, and 2 minutes after application. The descriptor “glidance” was evaluated on a scale at five levels: none, sparingly, moderately, quite, very.

The descriptor “glidance” is defined as being the ease of application of the product, its ability to cover a defined area and the capacity of the product not to drag on the skin during application. The notion of glidance is directly correlated to the SOFTNESS of the skin and acts favourably on the pleasantness and comfort of the product.

The sheen was evaluated 2 minutes after penetration, on a scale at five levels: none, sparingly, moderately, quite, very.

Results:

| | A | B (invention) |
|---|---|---|
| Glidance on application | 2/10 experts: None | 2/10 experts: Quite |
| and finally | 8/10 experts: Sparingly | 8/10: Very |
| Final skin sheen | 2/10 experts: Quite | 8/10 experts: None |
| | 8/10 experts: Very | 2/10 experts: Sparingly |

In conclusion, the composition according to the invention is more glidant (softer) on application and leaves the skin softer and less shiny after penetration. Moreover, composition A is unstable (creaming) after 15 days of storage at 37° C., whereas composition B according to the invention is stable.

### EXAMPLE 2

### Amounts Expressed in g % of Active Material

| | C | D | E |
|---|---|---|---|
| Octyltriethoxysilane | 2 | 2 | 2 |
| Ethylenediamine/stearyl dilinoleate | 8 | — | — |
| copolymer (Uniclear ® 100 VG - Arizona | | | |
| Chemical) | | | |
| Fumed silica | — | 8 | — |
| Poly C₁₀₋₃₀ alkyl acrylate (Intelimer | — | — | 8 |
| IPA 13-1) | | | |
| Cyclopentadimethylsiloxane | qs 100 | qs 100 | qs 100 |

Hair treated at room temperature with compositions C, D and E in leave-in mode are smooth and soft, without having a greasy look or feel.

1. Cosmetic composition comprising:
at least one lipophilic thickener and
at least one silane corresponding to formula (I) below and/or oligomers thereof:
R₁Si(OR₂)_{z}(R₃)ₓ(OH)_{y} (I)
in which:
R₁ is a linear or branched, saturated or unsaturated C₁-C₂₂ hydrocarbon-based chain, which may be substituted with a hydroxyl group, a thiol group, an aryl group, which is substituted or unsubstituted; R₁ optionally being interrupted with a heteroatom selected from the group consisting of O and S or a carbonyl group (CO),
R₂ and R₃, which may be identical or different, represent a linear or branched alkyl group comprising from 1 to 6 carbon atoms,
y denotes an integer ranging from 0 to 3, and
z denotes an integer ranging from 0 to 3, and
x denotes an integer ranging from 0 to 2,
with z+x+y=3,
the silane(s) of formula (I) and/or oligomers thereof being present in an active material content ranging from 2% to 20% by weight relative to the total weight of the composition.

2. Composition according to the claim 1, wherein R₂ represents an alkyl group comprising from 1 to 4 carbon atoms, and R₃ represents an alkyl group comprising from 1 to 4 carbon atoms.

3. Composition according to claim 1, wherein R₁ represents an alkyl group comprising from 7 to 18 carbon atoms.

4. Composition according to claim 1, wherein z ranges from 1 to 3.

5. Composition according to claim 1, which comprises at least one silane chosen from octyltriethoxysilane, dodecyltriethoxysilane, octadecyltriethoxysilane and hexadecyltriethoxysilane.

6. Composition according to claim 1, which comprises at least octyltriethoxysilane.

7. Composition according to claim 1, wherein the silane(s) of formula (I) and/or oligomers thereof are present in an active material content ranging from 5% to 15% by weight relative to the total weight of the composition.

8. Composition according to claim 1, wherein the lipophilic thickener is chosen from semi-crystalline polymers, mineral lipophilic thickener(s), organic lipophilic thickeners other than semi-crystalline polymers, and mixtures thereof.

9. Composition according to claim 1, wherein the lipophilic thickener is chosen from semi-crystalline polymers, clays and silicas, and mixtures thereof

10. Composition according to claim 1, wherein the organic lipophilic thickener is non-silicone.

11. Composition according to claim 1, wherein the lipophilic thickener(s) are present in proportions ranging from 0.1% to 50% by weight relative to the total weight of the composition.

12. Composition according to claim 1, which contains at least one additional fatty substance, preferably chosen from C₆-C₁₆ hydrocarbons, hydrocarbons containing more than 16 carbon atoms, non-silicone oils of animal origin, plant oils of triglyceride type, synthetic triglycerides, fluoro oils, fatty alcohols, esters of fatty acid and/or of fatty alcohol other than triglycerides, and plant waxes, non-silicone waxes and silicones, and mixtures thereof.

13. Composition according to claim 1, which is aqueous.

14. Composition according to claim 1, which is anhydrous.

15. Cosmetic composition, which is obtainable by mixing at least one silane as described in claim 1 and at least one lipophilic thickener.

16. Cosmetic process for treating keratin materials, in which a cosmetic composition as defined in claim 1 is applied to the keratin materials.

17. Use of a cosmetic composition as defined in claim 1, in the cosmetic or dermatological field.

18. Composition according to the claim 1, wherein R₂ represents ethyl, and R₃ represents methyl or ethyl groups.

19. Composition according to-claim 1, wherein R₁ represents octyl.

20. Composition according to_claim 1, wherein z is equal to 3.
